(19) United States
(12) Patent Application Publication
(10) Pub. No.: US 2009/0035746 A1
(43) Pub. Date: Feb. 5, 2009

(54) Device and Method for Preparing a Sample for an Analysis and Device and Method for Analyzing a Sample

(76) Inventors: Thomas Ehben, Weisendorf (DE); Christian Zilch, Leipzig (DE)

Correspondence Address: HARNESS, DICKEY & PIERCE, P.L.C., P.O. BOX 8910, RESTON, VA, 20195, US

(21) Appl. No.: 11/922,879

(22) PCT Filed: Jun. 21, 2006
(86) PCT No.: PCT/EP2006/063399

§ 371 Date: Dec. 26, 2007

(30) Foreign Application Priority Data
Jun. 27, 2005 (DE) 10 2005 029 809.5

(51) Int. Cl. C12Q 1/00; C12M 1/00; C12N 13/00; C12P 19/34

(52) U.S. Cl. 435/4; 435/283.1; 435/173.1; 435/91.2

(57) ABSTRACT

A device and method are disclosed for preparing a sample for an analysis and a device and method are disclosed for analyzing a sample. In at least one embodiment, a device is disclosed for preparing a sample for analysis including means for binding at lest one biological structure of the sample, means for releasing at least one biological molecule contained in the at least one structure, and means for binding the at least one released molecule. The means for binding the at least one structure and the means for binding the at least one molecule can move.

### PRIORITY STATEMENT

This application is the national phase under 35 U.S.C. § 371 of PCT International Application No. PCT/EP2006/063399 which has an International filing date of Jun. 21, 2006, which designated the United States of America and which claims priority on German Patent application 10 2005 029 809.5 filed Jun. 27, 2005, the entire contents of which are hereby incorporated herein by reference.

### FIELD

At least one embodiment of the present invention relates to a device and a method for preparing a sample for an analysis and to a device and a method for analyzing the sample.

### BACKGROUND

Biotechnology and genetic engineering have increasingly gained in importance in recent years. One basic task in biotechnology and genetic engineering is to detect biological molecules such as DNA (deoxyribonucleic acid) or RNA (ribonucleic acid), proteins, polypeptides, etc. In particular molecules in which hereditary information is coded are of great interest for many medical applications. Detecting them, for example in a patient's blood sample, enables pathogens to be detected, which facilitates a diagnosis for a physician. DNA is a double helix constructed from two interlinked helical individual chains, so-called half strands. Each of these half strands has a base sequence, the hereditary information being defined by way of the order of the bases (adenine, guanine, thymine, cystosine). DNA half strands have the characteristic property of binding highly specifically only with very particular other molecules. Therefore, the docking of one DNA half strand to another DNA half strand presupposes that the respective molecules are arranged complementarily to one another.

This naturally prescribed principle can be used for the selective detection of molecules in a sample to be examined. The basic idea of a biochip sensor based on this principle consists in firstly so-called catcher molecules (oligonucleotides), being applied, for example by means of microdispensing, and immobilized on a substrate made of a suitable material, i.e. being permanently fixed at the surface of the biochip sensor. In this connection, it is known to immobilize biomolecules with thiol groups (SH groups) at gold surfaces.

A corresponding biochip sensor having a substrate and catcher molecules which are bound thereto and are sensitive for example to a particular DNA half strand to be detected is usually used for examining a sample, normally in the form of a liquid, with regard to the presence of the DNA half strand. For this purpose, the sample which is to be examined with regard to the presence of a specific DNA half strand is to be brought into operative contact with the immobilized catcher molecules. If a catcher molecule and a DNA half strand to be examined are complementary to one another, then the DNA half strand hybridizes to the catcher molecule, i.e. it is bound thereto. If, on account of this binding, the value of a metrologically detectable physical quantity changes in a characteristic manner, then the value can be measured and the presence or absence of a DNA half strand in the sample to be examined can be ascertained in this way.

The principle described is not restricted to the detection of DNA half strands. Rather, further combinations of catcher molecules applied on the substrate and molecules to be detected in a sample to be examined are known. Thus, by way of example, it is possible to use nucleic acids as catcher molecules for peptides or proteins which bind in nucleic-acid-specific fashion. Furthermore, it is known to use peptides or proteins as catcher molecules for other proteins or peptides which bind the catcher peptide or the catcher protein. Electronic detection methods are often used for the detection of the binding effected between the catcher molecule applied on the substrate and the molecule to be detected which is present in the sample to be examined. Such detection methods are acquiring increasing importance in the industrial identification and assessment of new medicaments originating organically or from genetic engineering. The detection methods open up diverse applications for example in medical diagnosis, in the pharmacological industry, in the chemical industry, in foodstuffs analysis and also in ecology and foodstuffs technology.

In order to carry out an analysis of the type described above, for example in order to detect a specific DNA, a plurality of preparation steps by which DNA contained in the sample is extracted are generally required. The case of analysis of a patient's blood sample is discussed by way of example below, but the explanations can also be applied to other analysis methods.

The preparation steps to be carried out for analysis include a multiplicity of biochemical operations. Thus, it is necessary firstly to extract the constituents to be analyzed, for example bacteria or viruses, from the blood sample and to dispose of the rest of the blood sample. By way of a so-called lysis, the surrounding envelope of the viruses or bacteria is destroyed by way of a buffer solution and the DNA that is to be analyzed later is thus released. Since the blood sample generally contains too few copies of the DNA for a DNA detection, the DNA is replicated by means of a known reaction, the so-called polymerase chain reaction (PCR). Various possibilities for detecting the DNA and thus the pathogen are known in the further course of the analysis.

All the work steps can be carried out manually in a laboratory. Carrying out the work steps manually is complex and requires specially trained skilled workers. It is necessary to work with a multiplicity of reagents and other components. The process of preparing the sample and thus also the analysis of the sample become susceptible to errors and complicated as a result.

For a simplification and automation to the greatest possible extent, devices are known which comprise a reusable part, controller hereinafter, and a single-use part, cartridge hereafter. The sample is introduced into the cartridge and the cartridge is inserted into the controller. Within the cartridge, the sample is prepared and analyzed and the results are made available to a user, for example a laboratory technician or physician, by way of the controller. Such devices for preparing and/or analyzing the sample often have a multiplicity of mechanical and fluidic components. Thus, the reagents required for preparation and analysis are often stored in the controller. During the preparation and analysis of the sample, the reagents are pumped into the cartridge via fluidic interfaces. In this case, there is a risk of the cartridge being contaminated through the interfaces of the controller, which can lead to problems particularly in nucleic acid diagnostics.

There is the possibility of accommodating the required reagents for the reaction in the cartridge and mixing them as necessary with the sample in order to carry out the corresponding reaction. However, the required quantities of liquid generally lead to a large liquid volume and thus to a large cartridge. In particular, the sample is often transported through microfluidic channels by way of water transport, the water being moved within the channels by pumping.

The use of so-called magnetic beads is known for simplifying the preparation. A plurality of devices and methods are commercially available in this regard. The magnetic beads each comprise a ferromagnetic nanoparticle encapsulated in a polymer bead, for example, and having a diameter of a few micrometers. The magnetic beads can be provided externally with different affinity molecules or other suitable surface modifications. The magnetic beads are then suitable for binding specific biomolecules from a solution to their surface.

Typically, in a purification method, a suspension of magnetic beads is added to the sample to be separated in a test tube. A magnetic field is subsequently applied which separates the particles by accumulating them on one wall of the tube. The supernatant is discarded and the particles are then washed at least once. For this purpose, the magnetic field is removed first and the particles are suspended in a fresh solution. Renewed deposition on the vessel wall then takes place by reapplying the magnetic field. It is thus possible, after a plurality of washing steps, to suspend the molecules in a fresh solution by way of a buffer solution which separates the bound biomolecules from the magnetic beads. The magnetic beads are deposited again on the vessel wall, thereby making available the biomolecules in the supernatant solution. What is disadvantageous about the method described is the large amount of liquid required in each case, in the range of hundreds of microliters.

DE 101 11 520 B4 discloses a method for purifying biomolecules with the aid of magnetic particles, which enables in particular relatively small amounts of liquid to be purified as far as possible in an automated manner. It describes conveying the suspension with magnetic particles through a pipeline passing through a strong magnetic field. In this case, with suitable settings of diameter, flow rate and magnetic field strength, the magnetic particles are deposited on the wall of the pipeline when flowing through. The supernatant is discarded by emptying the pipeline or is collected in a receptacle.

The arrested particles can then be washed by rinsing with washing solutions. During the washing procedure, the magnetic particles can be held in the pipeline or be suspended and deposited again. The biomolecules are separated from the magnetic particles from the suspension by rinsing with a suitable buffer solution. In this case, the pipeline should be configured in such a way that small amounts of liquid of less than 50 μl can also be handled.

The method described is suitable in particular for purifying DNA or RNA. The DNA or RNA available in solution at the end of the method can be introduced automatically into a corresponding analysis system. The automation can be effected by way of a pipeting robot, by way of example. If the DNA is to be detected via sequence-specific hybridization, it is proposed, moreover, to lead the pipeline additionally over a heating device in order to achieve denaturation of the DNA double strand. However, in order to analyze DNA using the method described, it is still necessary to extract the DNA from the sample by way of method steps which have not been described.

DE 101 11 457 A1 discloses a diagnosis device for application in biochemical analysis, in which a smart card for measurement is inserted into a controller. A plurality of reagent channels are shaped in the smart card, the channels containing reagents required for the process in a predosed amount. Each reagent channel is provided with a water inlet through which water can be introduced into the smart card from the controller. Via a sample channel connected to the reagent channels, the sample to be analyzed is introduced into the smart card through a sample port. A sensor module is arranged in the sample channel, catcher molecules being arranged on the sensor module. By way of example DNA molecules in the sample can be detected by way of the sensor module. An outlet is present downstream of the sensor module at the sample channel, through which outlet the sample is conveyed after flowing past the sensor module into the controller for disposal. For conveying the sample, at least one pump is provided in the controller.

Liu et al., in Anal. Chem. (2004) 76, 1824-1831, disclosed a fully integrated biochip in which cells are extracted from a sample by way of binding to magnetic beads. The sample is pumped into a chamber where magnetic beads are added. By way of an antibody coating of the magnetic beads, the blood cells sought are bound to the magnetic beads and, upon being pumped further into a PCR chamber, are retained by a magnet arranged there. Afterward, a PCR is carried out and the PCR product resulting from this is pumped into a hybridization chamber and detected there.

Lee et al., in Appl. Phys. Lett. (2004) 85, 1063-1065, described a device comprising a microelectromagnet matrix. The matrix makes it possible to move magnetic beads within a microfluidic channel. In this case, the individual magnets of the matrix are progressively switched on and off, such that a net force is exerted on the magnetic beads.

U.S. Pat. No. 6,605,454 B2 describes a microfluidic device which uses magnetic beads for separating nucleic acids from a sample liquid. Firstly, cells contained in the sample liquid are subjected to lysis and the nucleic acids thus released are bound by way of the magnetic beads. The magnetic beads with the bound nucleic acids are held back by applying a magnetic field, while the rest of the sample liquid is pumped into a waste channel. The nucleic acid is separated from the rest of the sample liquid in this way and can be analyzed further.

WO 01/41931 A2 describes a microfluidic system having integrated sample preparation and detection. In this case, a sample is firstly pumped into a lysis chamber, in which cell disruption is carried out in order to release the DNA. In a subsequent separation chamber, the DNA is separated from the sample. For this purpose, use is made of paramagnetic magnetic beads which bind the DNA and are held back on the chamber wall by way of a magnetic field. After separation and washing, the DNA is pumped into a further chamber, in which an amplification reaction is carried out. Finally, the DNA is detected in a detection chamber. The transport of cells and DNA between the various chambers is effected by way of pumping and the chambers are connected by microchannels.

Gijs, in Microfluid. Nanofluid. (2004) 1, 22-40, summarized diverse applications for magnetic beads. It is pointed out, inter alia, that solutions are known in which magnetic particles can be moved a number of millimeters in microfluidic channels. The solutions described can be used for example in so-called Lab-on-a-Chip systems.

Smistrup et al., in the Journal of Magnetism and Magnetic Materials (2005) 293, 597-604, described simulations and experiments concerned with the magnetic separation of magnetic systems. In this case, magnetic beads are held back from a flowing liquid by way of a magnetic field.

Choi et al., in Lab Chip (2002) 2, 27-30, described an integrated microfluidic biochemical detection system for protein analysis. In this case, antibody-coated magnetic beads are used and immobilized by a magnetic field. Target antigens are subsequently pumped through the microfluidic channel, such that they can attach to the immobilized magnetic beads. After a magnetic field has been switched off, the magnetic beads are transported from the chamber by way of liquid flow.

WO 2004/078316 A1 describes a transport device for magnetic beads. In this case, coils are arranged in the region of a capillary chamber. The configuration of the resulting magnetic fields makes it possible to transport the magnetic beads through the chamber.

### SUMMARY

At least one embodiment of the present invention, a device and a method are disclosed for preparing a sample for an analysis and a device and a method for analyzing a sample in which the devices are constructed in a simple manner and manual work steps can be dispensed with to the greatest possible extent.

The device-related objects are achieved by way of devices having the features of claim 1 and of claim 32, respectively. The method-related objects are achieved by way of methods having the features of claims 42 and 64, respectively.

According to at least one embodiment of the invention, the device for preparing a sample comprises the following features:
means for binding at least one biological structure of the sample,
means for releasing at least one biological molecule contained in the at least one structure, and
means for binding the at least one released molecule,
at least one magnetic field generator embodied in such a way that the means for binding the at least one structure and the means for binding the at least one molecule can be moved thereby.

The term “biological structure” should be understood hereinafter to mean in particular bacteria, cells and viruses. However, it can also mean other biological structures of the sample, such as, for example, proteins, peptides, spores, chromosomes, protozoa, or other constituents of the sample that are to be analyzed. The term “biological molecule” should be understood hereinafter to mean primarily DNA, RNA, proteins, carbohydrates and lipids. In general, it should be understood to mean any types of molecules which are ultimately to be detected for analysis of the sample. These also include organic and inorganic toxins, for example. The explanations below relate in each case to one structure and one molecule contained therein or extracted therefrom. In general, however, a sample will contain a multiplicity of structures of different types, that is to say different bacteria, for example, which each contain molecules, that is to say DNA, for example. Likewise, in general there will be a multiplicity of specimens of each type of structure, and at least a portion of them will be prepared.

The means for binding the biological structure make it possible, in a simple manner, to extract the structure from the sample and supply it for further processing. Thus, firstly the sample structure to be analyzed is bound and then the biological molecule contained in the structure is released. The means for binding the released molecule make the latter accessible for further processing. Thus, by way of example, an analysis sought can subsequently be carried out.

It is possible to provide a plurality of means for binding different structures. Corresponding molecules are extracted from the structures by the releasing device and, if appropriate, bound by a plurality of means for binding the molecules. It is thus possible, in a simple manner, to detect a plurality of different structures within an analysis in the sample.

Likewise, in general a plurality of structures are bound by the means for binding the structure and supplied for further processing. This ensures that enough copies of molecules are present for a subsequent analysis.

In one advantageous embodiment of the invention, the means for binding the structure and the means for binding the molecule are embodied in movable fashion. Thus, both the structure and the molecule, after binding with the respective means, can be moved and processed further in a simple manner.

In one advantageous configuration of an embodiment of the invention, the means for binding a structure are embodied as a first substrate, which can be linked to the structure to form a substrate-structure complex. The means for binding the molecule are embodied as second substrate, which can be linked to the molecule to form a substrate-molecule complex. The embodiment of the respective means as substrates constitutes a simple possibility of realization for the respective binding means.

In one advantageous configuration of an embodiment of the invention, the substrates in each case comprise at least one magnetic bead. By way of the magnetic property, the substrate-structure and substrate-molecule complexes can be manipulated particularly simply according to known methods within the device. In particular, the steps of introducing the sample into the device, separating the structures from the sample, releasing the molecules and processing the molecules further can thus be realized in a simple manner.

The magnetic beads can be adapted, by coating their surface or by applying catcher molecules, in a simple manner to the requirements of the preparation or analysis to be carried out. Thus, what can be achieved for example by way of oligonucleotides on the surface of the magnetic beads is that these only bind the type of DNA sought and thus make it accessible to further processing. The magnetic beads can combine with cells by way of antibodies on the surface.

A configuration of an embodiment of the invention such that the means for binding the structure and the means for binding the molecule are embodied as a third substrate is particularly advantageous. The substrate can then be linked both to the structure to form a substrate-structure complex and to the molecule to form a substrate-molecule complex. Combining both binding means on a single substrate simplifies the device further, since fewer components are required. Thus, in a corresponding method, firstly the structure is linked to the substrate to form the substrate-structure complex and then the means for releasing the biological molecule are added. The released molecule is then linked to the substrate to form the substrate-molecule complex and can be processed further for the analysis. If a plurality of copies of molecules are required for detection, then it is possible to extract these in a simple manner by increasing the number of substrates of identical type from the sample, if present.

If a plurality of different structures are to be detected, corresponding substrates of different types should be provided, such that for each structure to be detected, one type of substrate which can bind with the structure and the molecule contained therein is present in the device.

In this case, too, it is advantageous to embody the third substrate as magnetic beads. Magnetic beads can be saved by virtue of the multiple functionalization. Only one type of magnetic beads is required for one structure to be detected. By way of magnetic beads with differing functionalization, a plurality of structures and molecules can be processed simultaneously in the device.

In one advantageous configuration of an embodiment of the invention, the device comprises a number of process chambers which contain at least temporarily the means for binding the structure, the means for releasing the molecule and the means for binding the molecule. By separating the process into a plurality of process chambers, it can be ensured that only those parts of the sample which are to be processed further are in each case supplied to the respective process steps.

In an embodiment, the process chambers employs as a preparation chamber for using the means for binding the structure. It further employs a structure disruption chamber for using the means for releasing the molecule and the means for binding the molecule. Thus, the sample can be introduced into the preparation chamber, whereupon the sample structures to be analyzed are bound by the magnetic beads. The magnetic beads are then moved into the structure disruption chamber, where the corresponding structures are dissolved and the molecule is released. The released molecule is bound by the substrate and is available for further analysis.

It is particularly advantageous to store the means for binding the structure in the preparation chamber and to store the means for releasing the molecule and the means for binding the molecule in the structure disruption chamber. The use of dry reagents is particularly advantageous in this case. Thus, at the beginning of the method, the corresponding process chambers can be flooded with water, for example, and the reagents can thus be dissolved. After the sample has been added, the sample is prepared as far as possible in an automated manner.

The preparation chamber can either be contained with the remaining process chambers in a continuous unit. As an alternative, it is possible to embody the preparation chamber as a syringe, for example, and to carry out the preparation of the sample in one of the. The means for binding the structure are correspondingly stored in the syringe. It is only after preparation that the sample is introduced into the unit with the rest of the process chambers and thus supplied for further preparation.

In one advantageous configuration of an embodiment of the invention, the microchannels are dimensioned in such a way that both the substrate-structure complex and the substrate-molecule complex can be transported through them and that a disturbing exchange of other substances between the process chambers is prevented while the process steps are carried out. Although an exchange of liquids between the process chambers will always take place through the inherently opened microchannels, by way of sufficiently small dimensioning it is possible to permit only a very small exchange. The latter should be chosen to be so small that, for the duration of a preparation or analysis operation, the process steps that proceed are not disturbed by the exchange.

In one advantageous configuration of the device, the latter comprises a single-use unit and a controller. The process chambers are contained in the single-use unit. This primarily enables simple handleability of the device. Thus, the sample can be introduced into the single-use unit and the latter, according to a procedure known per se, can be inserted into the controller. The preparation of the sample for the analysis takes place in the process chambers of the single-use unit.

An advantageously embodied device contains at least one device for moving and/or fixing the substrate-structure complex and the substrate-molecule complex. What can thereby be achieved in a simple manner is that the substrate-structure complexes can be moved from the preparation chamber into the structure disruption chamber and can be fixed there.

The device according to an embodiment of the invention for analyzing the sample comprises a device of the type described above. Moreover, at least one device for detecting the biological molecule is present.

A device is advantageous such that the at least one device for detecting the molecule are arranged at least partly in a detection chamber of the device. The substrate-molecule complexes, after the release of the molecule and binding to the substrates, can be moved into the detection chamber of the device and analyzed there in a simple manner.

In one advantageous configuration of the device, the at least one device for detecting the molecule comprise a detection unit and further means for binding the molecule. The further means for binding the molecule make it possible for the substrate-molecule complexes to be bound in the detection chamber and detected by the detection unit.

In a particularly advantageous embodiment of the device, the detection unit comprises a magnetoresistive sensor. The magnetic beads present in the substrate-molecule complexes can be detected by the magnetoresistive sensor in a simple manner. A suitable magnetoresistive sensor is a GMR sensor (giant magnetoresistance), for example, such as is known from present-day hard disk technology and is available in large numbers and with a small size. As an alternative, it is also possible to use a TMR sensor (tunneling magnetoresistance), such as is used for example in magnetic main memories (MRAM) for computers.

The method according to an embodiment of the invention for preparing the sample by way of a device according to the type described above comprises the following method steps:
binding of the at least one biological structure of the sample by the means for binding the structure,
moving the bound biological structure by way of the at least one magnetic field generator,
releasing the at least one biological molecule contained in the bound structure,
binding of the released molecule by the means for binding the molecule.

With one of the devices described, the method for preparing the sample can be carried out as far as possible in an automated manner. By virtue of fewer manual steps, in particular the cleanliness of the process is increased and incorrect analyses are thus avoided.

The method according to an embodiment of the invention for analyzing the sample comprises the following method steps:
preparing the sample by a method according to the type described above, and
detecting the molecule.

The method described can be carried out as far as possible in an automated manner with the corresponding device.

### BRIEF DESCRIPTION OF THE DRAWINGS

Further advantages of the invention are explained in the example embodiments described below with reference to the figures, in which:

FIG. 1 shows a schematic illustration of a cartridge with various process chambers,

FIGS. 2 to 4 show various embodiments of a cartridge and of a controller,

FIG. 5 shows a schematic flowchart of a method for analyzing a sample.

### DETAILED DESCRIPTION OF THE EXAMPLE EMBODIMENTS

Various example embodiments of the invention are described below. This is based in each case on an example of a device for preparing and analyzing a patient's blood sample. Previously defined cells are sought in the blood sample. The device is specifically tailored to the cells. The DNA is extracted from the cells and detected.

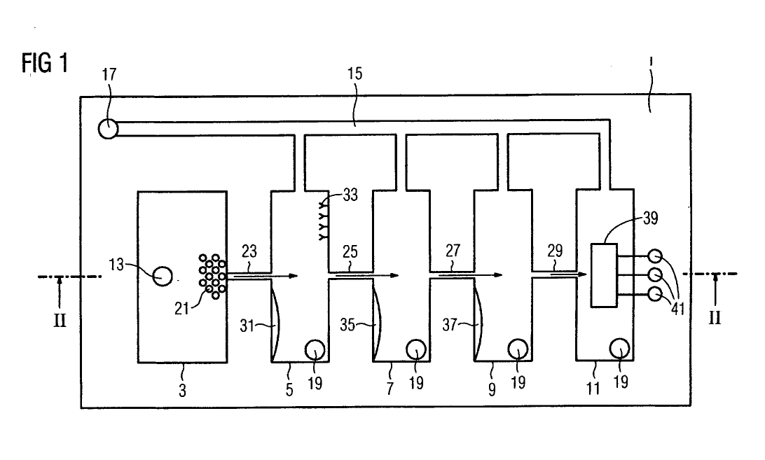

FIG. 1 schematically illustrates a cartridge 1. It comprises a preparation chamber 3, a structure disruption chamber 5, a washing chamber 7, an amplification chamber 9 and a detection chamber 11. The preparation chamber 3 includes a filling opening 13, via which the sample to be examined can be introduced into the preparation chamber 3 by way of a syringe, for example. Apart from the preparation chamber 3, each of the process chambers is connected to an opening 17 via a microchannel 15. Water is introduced into the process chambers via the opening 17 in a manner known per se.

Apart from the preparation chamber 3, each process chamber has a venting opening 19 closed off by a membrane, for example. It can thus be ensured that gas can leave the process chambers, but liquids cannot leave them. A venting opening is not necessary at the preparation chamber 3 if, when the sample is introduced, the rest of the process chambers have not yet been filled with water. In this case, the preparation chamber 3 is vented via the structure disruption chamber 5. If flooding with water is intended to be effected before the introduction of the sample in the process sequence, then a corresponding venting opening should also be provided at the preparation chamber 3.

Magnetic beads 21 are stored beforehand in dry form in the preparation chamber 3. The preparation chamber 3 is flooded by the introduction of the sample, such that the magnetic beads 21 are suspended and dispersed in the sample.

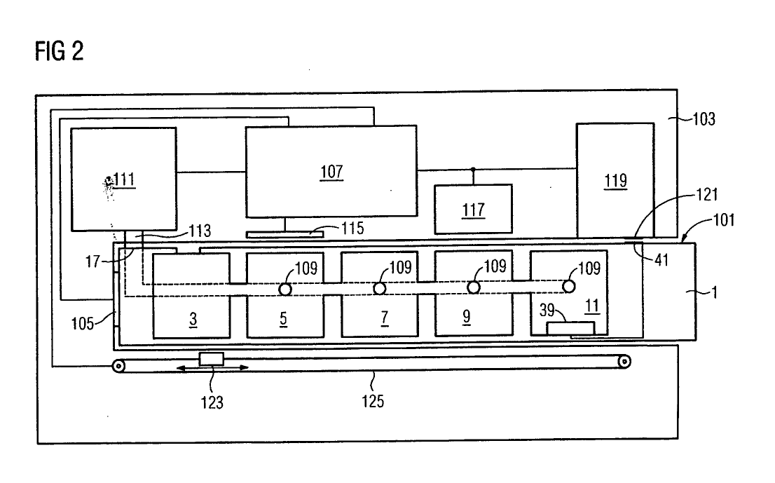

The isolation of the cells sought from the sample takes place by way of magnetic beads 21 having antibodies arranged on their surfaces. By way of the antibodies, the cells are bound to the magnetic beads 21 and immobilized on their surface. Both monoclonal and polyclonal antibodies can be used in this case. In this case, monoclonal antibodies are only directed toward one cell type to be bound, whereas polyclonal antibodies comprise a mixture by which different cell types can be bound. However, it is also possible to use less specifically binding molecules such as, for example, proteins for binding bacterial liposaccharides. For optimally intermixing the sample and the magnetic beads 21 and accelerating the linking reaction, the magnetic beads 21 are moved to and fro in the preparation chamber 3. Various mechanisms for moving the magnetic beads 21 within the cartridge 1 are illustrated in FIGS. 2 to 4.

The process chambers are interconnected by microchannels 23 in accordance with the order of the process steps that proceed. The microchannels 23, 25, 27 and 29 are dimensioned in such a way that an interfering exchange of liquid between the process chambers is prevented to the greatest possible extent during the entire duration of preparation and analysis and has no interfering influence. On the other hand, the microchannels 23, 25, 27 and 29 are large enough to permit magnetic beads 21 with bound structures or molecules to pass through. Complexes in which a plurality of magnetic beads 21 are bound to a structure or a plurality of structures are bound to a magnetic bead 21 can also pass through the microchannels 23, 25, 27 and 29. The same applies to the complexes composed of magnetic beads 21 and molecules. The diameter is likewise large enough to permit conglomerates to pass through in which a plurality of magnetic beads 21 and a plurality of structures or molecules agglomerate. Consequently, the diameter of the microchannels 23, 25, 27 and 29 will typically be of the order of magnitude of several μm.

As an alternative, it is possible for the microchannels 23, 25, 27 and 29 to have larger dimensions and to be closed off by valves. The valves are briefly opened in order to allow the magnetic beads 21 with bound cells or DNA to pass through, and are then closed again.

After the magnetic beads 21 have combined with the cells sought from the sample, the corresponding complexes are moved through the microchannel into the structure disruption chamber 5. Suitable reagents 31 (lysis buffer) that enable a structure disruption of the cells are stored in the structure disruption chamber 5. The DNA molecules lying within the cells are thereby released. This process is supported by a slight heating. In order to obtain the heating, a Peltier element or a heating element is provided in the controller, which is described in further detail below. The lysis buffer known per se is present in dry form in the structure disruption chamber 5 and is dissolved by the flooding with water at the beginning of the method.

The cell-binding properties of the antibodies on the magnetic beads 21 are no longer required in the further course of preparation and analysis and can be removed. For this purpose, a protease enzyme is admixed with the lysis buffer and detaches the antibodies from the surface of the magnetic beads 21 and destroys them. This prevents cell residues that might have an interfering influence on the analysis process from attaching to the magnetic beads 21.

The DNA contained in the cells is released by the structure disruption. The DNA is intended to be analyzed in the further course of the method. The DNA molecules are generally too large for an analysis. The DNA molecules are therefore comminuted. For this purpose, a restriction enzyme is added to the lysis buffer and cleaves the DNA either at specific sites or nonspecifically, depending on the enzyme used. In this case, the degree of comminution of the DNA is determined by the concentration of the restriction enzyme in the lysis buffer. As an alternative, it is possible to comminute the DNA by mechanical stress. In this case the magnetic beads 21 to which the DNA is bound can be caused to effect rapid movements in the structure disruption chamber 5. The resulting agitating effect comminutes the DNA molecules.

For the later analysis it is likewise necessary to separate the DNA that is still present as a double strand. This operation is referred to as denaturation and takes place by way of sodium hydroxide solution added to the lysis buffer and brief heating of the lysis buffer to approximately 40° C.

As an alternative, it is possible to leave the DNA as a double strand and not to denature it until at a later point in time. In this case, sodium hydroxide solution is not added to the lysis buffer.

In a large number of analysis operations, the DNA sought is only present in a very small number in the sample. This occurs, for example when examining blood samples with regard to bacteria or viruses. In such blood samples, there is much more human DNA (eukaryotic DNA) in the sample than for example viral or bacterial DNA (prokaryotic DNA). In these cases it is important to separate the eukaryotic DNA in order to avoid interference with the further process steps. For this purpose, specific antibodies 33 that bind histones are immobilized on a wall of the structure disruption chamber 5. Since histones only occur in eukaryotic DNA, only this form of DNA is immobilized on the wall. The prokaryotic DNA can be supplied for further preparation and analysis.

As an alternative, it is possible to apply the specific antibodies 33 to the surfaces of the magnetic beads 21 and in this way only supply the eukaryotic DNA for further analysis.

In order to separate the DNA from the mixture present in the structure disruption chamber 5, the comminuted DNA strands are bound to the magnetic beads 21. For this purpose, the magnetic beads 21 have a further binding property in the form of oligonucleotides or a silane coating of the surface. While the oligonucleotides bind specifically with the DNA to be analyzed, the silane binds all DNA strands nonspecifically. Moreover, only denatured DNA can bind to the oligonucleotides, while DNA double strands can also be bound to the silanized surface. If a plurality of types of DNA, for example of different types of bacteria, are intended to be detected during the analysis, then it is also possible, besides the use of the silane coating, to use magnetic beads 21 functionalized with different oligonucleotides. Oligonucleotides complementary to the DNA strands to be examined are then arranged on the surfaces of the respective magnetic beads 21. It is thus possible, in a simple manner, to prepare a plurality of DNA molecules.

The washing chamber 7 is provided to eliminate cell residues and other contaminants that may be present. The complexes composed of magnetic beads 21 and DNA are moved through the microchannel 25 into the washing chamber 7. Chaotropic salts 35 are stored in the washing chamber 7, which salts are initially present in dry form and dissolve as a result of the washing chamber 7 being filled with water at the start of the process. In order to support the washing operation, the magnetic beads 21 are caused to effect a circulating movement.

For complex preparation or analysis methods it is possible to provide a plurality of washing chambers which enable purification either successively or after different process steps.

In general, the DNA molecules bound to the magnetic beads 21 are not available in a sufficient number for a detection. For this reason, amplification takes place prior to the actual detection of the DNA. For this purpose, the magnetic beads 21 are moved into an amplification chamber 9, which is connected to the washing chamber 7 via the microchannel 27. An amplification, for example by way of a polymerase chain reaction (PCR), takes place in the amplification chamber 9. The amplification chamber 9 contains corresponding reagents in dry form which have been dissolved by way of water before the beginning of the process. The controller contains a Peltier element, by which thermal cycles can be carried out in the amplification chamber 9. The construction of the controller is shown schematically in FIG. 2.

When the temperature is increased, the DNA molecules are generally detached from the oligonucleotides of the magnetic beads 21. Denaturation additionally takes place in the case of DNA double strands. After the temperature has decreased, DNA primers, that is to say short DNA fragments, situated in the amplification chamber 9 bind to the single-stranded DNA molecules. These are supplemented by polymerase molecules to the full length, whereby DNA double strands arise. The DNA double strands are denatured by renewed heating. As a result of the temperature being decreased again, DNA primers again attach to the denatured DNA strands and are supplemented by the polymerase to full length. After a number of thermal cycles, the number of DNA molecules has increased significantly, such that enough DNA material is available for a detection. In the method described, it is also possible for different types of DNA to be amplified simultaneously, which is achieved by using different DNA primers.

It is possible to use the oligonucleotides immobilized on the magnetic beads 21 as DNA primers for the PCR. In that case it is no longer necessary for any DNA primers to be stored in the amplification chamber 9. The oligonucleotides on the magnetic beads 21 are extended by the polymerase. Since, in general, a multiplicity of oligonucleotides are immobilized on each magnetic bead 21 and DNA from the sample is bound only to a small percentage of the oligonucleotides, enough short oligonucleotides are available for the PCR cycles.

In order to detect the DNA, the DNA molecules bound to the magnetic beads 21 are moved through a microchannel into a detection chamber 11. Specific oligonucleotides are immobilized on a detection unit 39 in the detection chamber 11. The DNA molecules hybridize with the oligonucleotides and are thereby immobilized. The magnetic beads 21 likewise situated on the DNA molecules are likewise immobilized thereby. This operation can be accelerated by additional movement of the magnetic beads 21 within the detection chamber 11. In this case, the magnetic beads 21 can be moved circularly, on the one hand, and alternatively toward the oligonucleotides and away from them again by way of alternating movements. In order to increase the measurement accuracy and in order to lower the detection limit, the non-immobilized magnetic beads 21 are moved away from the oligonucleotides.

If the use of DNA primers was dispensed with during the PCR, the DNA strands bound to the magnetic beads 21 cannot hybridize since they are already complete double strands. In order to avoid this problem, the binding of free DNA strands to the extended oligonucleotides after the PCR is prevented by increasing the temperature.

The magnetic beads 21 are moved into the detection chamber 11, where the extended oligonucleotides hybridize directly with the oligonucleotides of the detection unit 39 without the DNA from the sample participating directly in the actual detection.

The detection of the DNA molecules sought takes place by detection of those magnetic beads 21 which are immobilized at that location of the chamber at which complementary oligonucleotides are arranged. For this purpose, the detection unit 39 includes a sensor that can detect the magnetic beads 21 on the basis of a specific property. This may be a magnetoresistive sensor, for example a GMR or TMR sensor. The immobilization of the magnetic beads 21 results in a local variation of the magnetic field. This variation brings about a corresponding change in resistance of the GMR sensor. Thus, the immobilized magnetic beads 21 can be determined by a measurement known per se of the resistance of the GMR sensor. By way of the known arrangement of the complementary oligonucleotides and the corresponding detection of the magnetic beads 21 immobilized on them, it is possible to determine what DNA is present in the sample. This in turn permits conclusions to be drawn about whether the cell sought is present in the sample. Electrical contacts 41 connected to the detection unit 39 are arranged on the cartridge 1. The measured values of the sensor can be transmitted to the controller via the contacts 41, which is explained with reference to FIG. 2.

As an alternative, it is possible for the magnetic beads 21 to be made visible by way of fluorescent dyes in an optical method known per se. For this purpose, the detection chamber 11 comprises transparent regions (not illustrated here) through which the accumulation of magnetic beads 21 on the specific oligonucleotides can be observed.

In an alternative embodiment of the invention, the sample is mixed with the magnetic beads 21 outside the device, whereby the corresponding cells to be analyzed combine with the magnetic beads 21. The sample prepared in this way is then introduced into the structure disruption chamber 5 directly via a corresponding filling opening.

In a further alternative embodiment of the invention, the microchannels 23, 25, 27 and 29 can be closed off by a respective valve. By way of the valve it is possible to ensure that no interfering exchange of liquid between the different process chambers can take place while the reaction is proceeding.

FIG. 2 shows a schematic illustration of the cartridge 1 in section along the line II. The cartridge 1 is inserted into a receptacle 101 of a controller 103, which is likewise illustrated in section. A stop 105 is formed in the rear part of the receptacle 101. This ensures that the cartridge 1 is inserted into the correct position.

The stop 105 is pressure-sensitive and connected to a control unit 107 that controls the process sequence for preparation and analysis. The control unit 107 automatically recognizes if a cartridge 1 has been inserted into the controller 103. An opening 109 is formed in each case in the structure disruption channel 5, the washing chamber 7, the amplification chamber 9 and the detection chamber 11, and water passes into the respective chamber through the opening at the beginning of the process.

The water originates from a supply container 111 arranged in the controller 103. The supply container 111 is connected to the control unit 107. After the cartridge 1 has been inserted into the receptacle 105, a maneuverable small tube 113 is pushed into the opening 17 and water from the supply container 111 is introduced into the process chambers. The dry reagents (not illustrated here) dissolve in the water and are available for the processing of the sample.

In the controller 103, a heating element 115 is arranged above the structure disruption chamber 5 and is connected to the control unit 107 for control purposes. The controller 103 additionally includes a Peltier element 117, which is arranged above the amplification chamber 9 and is likewise connected to the control unit 107. In order to evaluate the analysis, the controller 103 comprises an evaluation unit 119. A contact array 121 is arranged on the evaluation unit 119, and is in contact with the contacts 41 of the cartridge 1 after insertion. As a result, the data obtained by the detection unit 39 are available and can correspondingly be processed further. The evaluation unit 119 is connected to the control unit 107. In order to make the evaluated data accessible to a user, an interface to a computer or a monitor is provided, the interface not being illustrated here.

A permanent magnet 123 is arranged below the cartridge 1. The permanent magnet 123 is connected to a movement device 125, whereby it is movable both within the plane of the drawing and perpendicular thereto. The movement device 125 is connected to the control unit 107. It is possible, by the movement of the permanent magnet 123, to move the magnetic beads 21 within the cartridge 1 between the individual process chambers or, by the fixing of the permanent magnet 123, to retain them within one of the process chambers. It is additionally possible to move the permanent magnet 123 away from the process chambers, thereby preventing the magnetic field from influencing the magnetic beads 21. This is necessary, for example, in order that the magnetic beads 21 can be suspended and dispersed freely in the solution.

In addition, it is possible to move the permanent magnet 123 to and fro below each process chamber, such that the magnetic beads 21 and the cells or DNA strands bound thereto are moved to and fro. Circular movements that bring about an agitating effect are likewise possible. The movement within a chamber supports, on the one hand, the binding between magnetic beads 21 and the cells situated in the sample, the binding taking place in the preparation chamber 3, but also, on the other hand, the hybridization in the detection chamber.

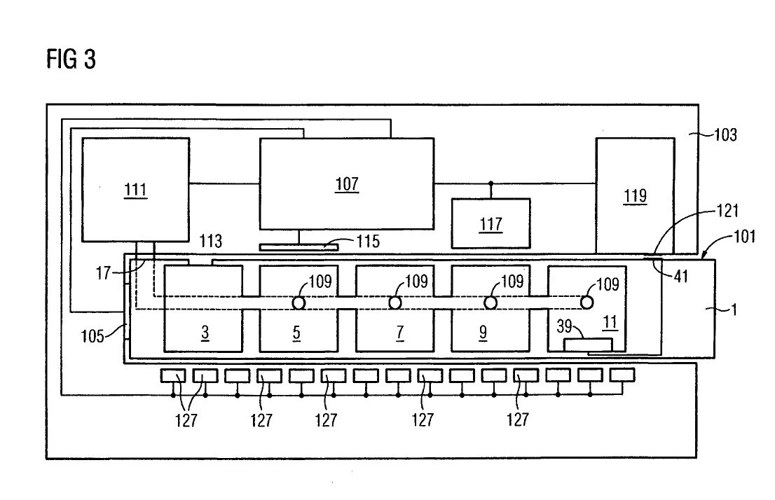

FIG. 3 shows the cartridge 1 in the controller 103 in an alternative embodiment. This embodiment differs from the one illustrated in FIG. 2 only by virtue of the configuration of the magnets for moving the magnetic beads 21. In the controller 103, a plurality of electromagnets 127 are arranged below the cartridge 1. Each electromagnet 127 is connected to the control unit 107. By sequentially switching the electromagnets 127 on and off, it is possible here, analogously to the example embodiment shown in FIG. 2, to move the magnetic beads 21 within the process chambers or between the latter. The rest of the movements described can likewise be performed.

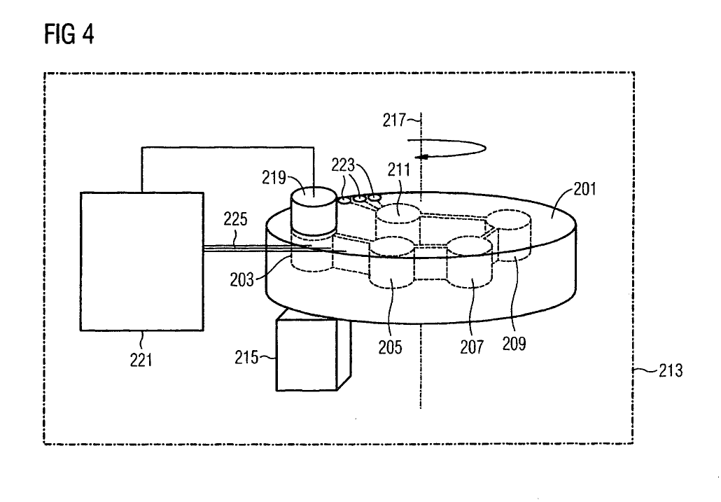

FIG. 4 schematically shows, in an alternative embodiment, a cartridge. 201 embodied in round fashion. In contrast to the example embodiment shown in FIG. 1, the process chambers 203, 205, 207, 209 and 211, which are only indicated here, are not arranged in a row one behind another, but rather along a circle arc. In the controller 213, a fixed magnet 215 is arranged below the cartridge 201, the magnet being formed as a permanent magnet or electromagnet. The cartridge 201 is mounted in a rotatable fashion after insertion into the controller 213. By rotating the cartridge 201 about the axis 217 it is now possible to move the magnetic beads 21 between the process chambers 203, 205, 207, 209 and 211. In this case, the magnetic beads 21 always remain fixed above the permanent magnet 215 while the cartridge 201 is moving.

A Peltier element 219 is arranged above the cartridge 201, and is fixed to a control unit 221. The temperature can thereby be controlled in the process chambers 203, 205, 207, 209 and 211. The cartridge 201 is rotated during the process, such that the magnetic beads 21 and thus also the cells or DNA successively run through all the process chambers 203, 205, 207, 209 and 211. A detection unit is arranged in the last process chamber 211, as in the exemplary embodiments described above, the detection unit being connected to contacts 223. The control unit 221 has measuring contacts 225 which, in an end position of the cartridge 201, that is to say when the process chamber 211 lies between the Peltier element 219 and the magnet 215, are in contact with the contacts 223. For the sake of better clarity, the supply container and the corresponding opening in the cartridge 201 have not been illustrated. The same applies to the filling opening and the microchannels for distributing the water into the process chambers 203, 205, 207, 209 and 211.

The device described can be used, in principle, for analyzing different types of samples. In this case, it is merely necessary to correspondingly adapt the binding properties of the magnetic beads 21 to the sample constituents to be detected and to provide corresponding replication or detection possibilities. The device described here for analyzing DNA and RNA can also be used for ribosomal nucleic acid. However, a protein analysis can also be carried out by adapting and supplementing the individual process steps.

In comparison with known analysis methods and devices for DNA analysis, the device described here has the advantage that it enables the analysis steps to be automated to the greatest possible extent. Thus, apart from introducing the sample into the cartridge and inserting the cartridge in the controller, there is no need to carry out any manual step until the results are obtained. In addition, the method can be parallelized, such that different DNA can be analyzed in one analysis procedure. For this purpose, different oligonucleotides are correspondingly arranged in the detection chamber.

A further advantage of the device described here is the integration of virtually the entire analysis process into a compact device that is simple to handle. By virtue of its simple construction in comparison with known solutions, without movable parts, the cartridge can be produced particularly simply and cost-effectively. In the controller, it is possible to dispense with a complex mechanism for controlling liquids within the cartridge, since the cells and the DNA are only moved by way of magnetic forces.

A plurality of washing chambers are arranged in an alternative embodiment of the cartridge, the washing chambers being constructed analogously to the washing chamber 7 illustrated in FIG. 1. Thus, a washing chamber is arranged between the preparation chamber 3 and the structure disruption chamber 5 in order to be able to purify the sample prior to structure disruption. It is analogously possible to arrange a plurality of washing chambers in succession, such that a plurality of washing operations can be performed in succession.

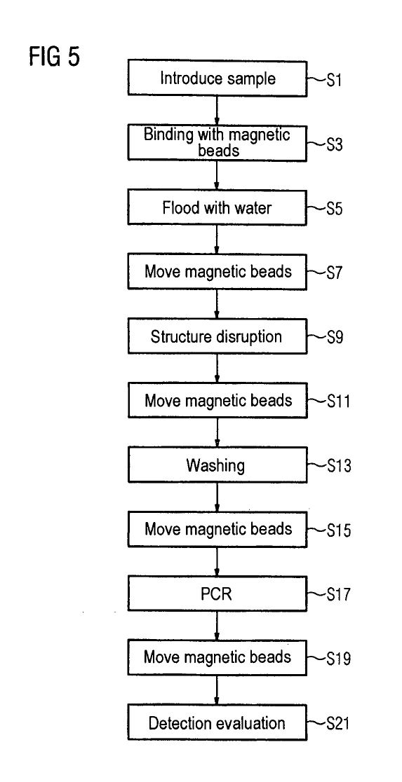

FIG. 5 shows a schematic flowchart of an analysis method using one of the devices described above. In a first method step S1, the patient's sample is introduced into the cartridge through the filling opening. The cartridge is thereupon inserted into the controller, whereby the analysis process starts automatically. In a second method step S3, in the preparation chamber, the magnetic beads stored there bind with the sample cells to be examined.

In this case, the magnetic beads in the solution are moved to and fro by suitable manipulation of the magnetic field in order to accelerate the operation. In a third method step S5, the process chambers of the cartridge are filled with water and the reagents stored there in dry form are dissolved. In a fourth method step S7, the magnetic beads are moved into the structure disruption chamber by the magnetic field. A valve possibly arranged in the microchannel between the preparation chamber and the structure disruption chamber is briefly opened for this purpose. In a fifth method step S9, by way of the lysis buffer that is stored in the structure disruption chamber and is present in solution as a result of the flooding with water, the cells bound to the magnetic beads are dissolved and the DNA that they contain is released. The DNA is denatured by briefly heating the lysis buffer.

As an alternative, sodium hydroxide solution can also be added to the lysis buffer, whereby denaturation is likewise achieved. The released DNA molecules bind to the magnetic beads. At the same time, the antibodies are detached from the surface of the magnetic beads by a protease enzyme in the lysis buffer, such that residues of the cell structures are also no longer attached to the magnetic beads. Consequently, the magnetic beads are only linked to the DNA molecules of the cells to be analyzed.

In a sixth method step S11, the magnetic beads with DNA molecules are moved through a microchannel into the washing chamber of the device. In a seventh method step S13, residues of cells and other contaminants that are possibly present are washed out. In an eighth method step S15, the magnetic beads are moved into the amplification chamber. In a ninth method step S17, a polymerase chain reaction is carried out in the amplification chamber and the DNA of the cells to be examined is thereby replicated. In order to carry out the chain reaction, a plurality of thermal cycles between two temperatures are carried out in the amplification chamber by way of a Peltier element.

In a tenth method step S19, the magnetic beads and the DNA fragments bound thereto are moved through a microchannel into the detection chamber of the device, in which hybridization of the DNA fragments with oligonucleotides arranged in the detection chamber takes place in an eleventh method step S21. In this case, on account of the specific binding properties, only those DNA fragments which are intended to be analyzed are attached to the oligonucleotides. Consequently, these are tailored specifically to the analysis of a specific cell type.

In an alternative method, it is possible to provide even further purifying steps analogously to method step S13, for example in a further washing chamber arranged between the preparation chamber and the structure disruption chamber. It is additionally possible to arrange a plurality of washing chambers in succession in order to be able to perform a plurality of washing steps in succession.

The method described above only relates to one type of DNA to be detected, for example of a specific virus. However, the method steps can also be parallelized in such a way that different types of DNA can be detected. In that case it is necessary to provide correspondingly prepared magnetic beads and corresponding detection possibilities. It is likewise necessary to gear the PCR to a plurality of types of DNA.

It is likewise possible to include RNA in the analysis process. Before the amplification, the RNA is converted into so-called cDNA by reverse transcription and can then be replicated by way of PCR and detected by the detection unit.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A device for preparing a sample for an analysis, comprising:
means for binding at least one biological structure of the sample;
means for releasing at least one biological molecule contained in the at least one biological structure;
means for binding the at least one released biological molecule; and
at least one magnetic field generator to move the means for binding the at least one biological structure and the means for binding the at least one biological molecule.

2. The device as claimed in claim 1, wherein the means for binding the structure are embodied as a first substrate, linkable to the structure to form a substrate-structure complex, and wherein the means for binding the molecule are embodied as a second substrate, linkable to the molecule to form a substrate-molecule complex.

3. The device as claimed in claim 2, wherein the first substrate has a protein-binding property.

4. The device as claimed in claim 3, wherein the protein-binding property is embodied as at least one antibody, wherein the at least one antibody is directed toward the structure.

5. The device as claimed in claim 2, wherein the second substrate has a nucleic-acid-binding property.

6. The device as claimed in claim 5, wherein the nucleic-acid-binding property is embodied as an oligonucleotide complementary to the molecule.

7. The device as claimed in claim 5, wherein the nucleic-acid-binding property is embodied as silane.

8. The device as claimed in claim 2, wherein the first and second substrates each comprise at least one magnetic bead.

9. The device as claimed in claim 1, wherein the means for binding the structure and the means for binding the molecule are embodied as a third substrate, linkable both to the structure to form a substrate-structure complex and to the molecule to form a substrate-molecule complex.

10. The device as claimed in claim 9, wherein the third substrate has a protein-binding property and a nucleic-acid-binding property.

11. The device as claimed in claim 10, wherein the protein-binding property is embodied as at least one antibody and the nucleic-acid-binding property is embodied as an oligonucleotide, wherein the at least one antibody is directed toward the structure and the oligonucleotide is complementary to the molecule.

12. The device as claimed in claim 10, wherein the protein-binding property is embodied as antibody and the nucleic-acid-binding property is embodied as silane, wherein the at least one antibody is directed toward the structure.

13. The device as claimed in claim 9, wherein the substrate comprises at least one magnetic bead.

14. The device as claimed in claim 1, comprising a number of process chambers which contain at least temporarily the means for binding the structure, the means for releasing the molecule and the means for binding the molecule.

15. The device as claimed in claim 14, wherein the process chambers are embodied as a preparation chamber for using the means for binding the structure, and the device further comprising,
a structure disruption chamber for using the means for releasing the molecule and the means for binding the molecule.

16. The device as claimed in claim 15, wherein the means for binding the structure are stored in the preparation chamber and the means for releasing the molecule and the means for binding the molecule are stored in the structure disruption chamber.

17. The device as claimed in claim 1, further comprising means for replicating the molecule.

18. The device as claimed in claim 17, wherein the replicating means are stored in the structure disruption chamber of the device.

19. The device as claimed in claim 17, wherein the replicating means are stored in a further process chamber of the device, said further process chamber being embodied as an amplification chamber.

20. The device as claimed in claim 19, wherein at least one process chamber embodied as washing chamber is arranged between the structure disruption chamber and the amplification chamber.

21. The device as claimed in claim 14, wherein the process chambers are connectable by lines at least at times.

22. The device as claimed in claim 21, wherein lines are embodied as microchannels.

23. The device as claimed in claim 22, wherein at least one valve is included to close off at least one of the microchannels.

24. The device as claimed in claim 22, wherein the microchannels are dimensioned such that both the substrate-structure complex and the substrate-molecule complex are transportable through them and wherein a disturbing exchange of other substances between the process chambers is prevented while the process steps are carried out.

25. The device as claimed in claim 14, further comprising a single-use unit and a controller, the process chambers being contained in the unit.

26. The device as claimed in claim 25, wherein the means for binding the structure are embodied as a first substrate, linkable to the structure to form a substrate-structure complex, and wherein the means for binding the molecule are embodied as a second substrate, linkable to the molecule to form a substrate-molecule complex and wherein at least one magnetic field generator and the substrate-molecule complex are present in the controller.

27. The device as claimed in claim 26, wherein the at least one magnetic field generator is arranged in the controller.

28. The device as claimed in claim 27, wherein the at least one magnetic field generator is movable relative to the unit.

29. The device as claimed in claim 27, wherein the unit is movable relative to the at least one magnetic field generator.

30. The device as claimed in claim 27, wherein the at least one magnetic field generator is embodied as permanent magnet.

31. The device as claimed in claim 27, wherein the at least one magnetic field generator is embodied as electromagnet.

32. A device for analyzing a sample, comprising:
a device for preparing a sample as claimed in claim 1; and
means for detecting the at least one molecule.

33. The device as claimed in claim 32, wherein the means for detecting the molecule is arranged at least partly in the amplification chamber.

34. The device as claimed in claim 32, wherein the means for detecting the molecule is arranged at least partly in a process chamber of the device, said process chamber being embodied as detection chamber.

35. The device as claimed in claim 32, wherein the means for detecting the molecule comprise further means for binding the molecule.

36. The device as claimed in claim 35, wherein the further means for binding the molecule are arranged on a detection unit and the detection unit is arranged at least partly in the detection chamber.

37. The device as claimed in claim 35, wherein the further means for binding the molecule is embodied as at least one catcher probe.

38. The device as claimed in claim 37, wherein the substrate-molecule complex is immobilizeable by the catcher probe.

39. The device as claimed in claim 36, wherein the detection unit comprises a magnetoresistive sensor.

40. The device as claimed in claim 36, wherein the detection unit comprises an optical sensor.

41. The device as claimed in claim 36, wherein the detection unit comprises a gravimetric sensor.

42. A method for preparing a sample using a device including means for binding at least one biological structure of the sample, means for releasing at least one biological molecule contained in the at least one biological structure, means for binding the at least one released biological molecule, and at least one magnetic field generator to move the means for binding the at least one biological structure and the means for binding the at least one biological molecule, the method comprising:
binding the at least one biological structure of the sample with the means for binding the structure;
moving the bound at least one biological structure with the at least one magnetic field generator;
releasing the at least one biological molecule contained in the bound at least one biological structure; and
binding the released at least one biological molecule with the means for binding the molecule.

43. The method as claimed in claim 42, wherein the means for binding the structure are embodied as a first substrate, by which the structure is bound to form a substrate-structure complex, and the means for binding the molecule are embodied as a second substrate, by which the molecule is bound to form a substrate-molecule complex.

44. The method as claimed in claim 42, wherein the means for binding the structure and the means for binding the molecule are embodied as a third substrate, by which the structure is bound to form a substrate-structure complex and by which the molecule is bound to form a substrate-molecule complex.

45. The method as claimed in claim 43, wherein, after the binding of the structure to the substrate, the resulting substrate-structure complex is separated from the sample.

46. The method as claimed in claim 45, wherein the separation is effected by moving the substrate-structure complex into the structure disruption chamber.

47. The method as claimed in claim 46, wherein the substrate comprises at least one magnetic bead and a magnetic field is used for moving the substrate-structure complex.

48. The method as claimed in claim 47, wherein the magnetic field is generated by the at least one electromagnet of the device.

49. The method as claimed in claim 47, wherein the magnetic field is generated by the permanent magnet of the device.

50. The method as claimed in claim 49, wherein the permanent magnet is moved for moving the substrate-structure complex.

51. The method as claimed in claim 47, wherein the single-use unit of the device is moved relative to the permanent magnet.

52. The method as claimed in claim 42, wherein, after the molecules have been released, the means for binding the structure is removed from the substrate.

53. The method as claimed in claim 52, wherein the protein-binding property of the substrate is removed by use of a protease enzyme.

54. The method as claimed in claim 52, wherein the released molecules are comminuted.

55. The method as claimed in claim 54, wherein each of the molecules is a nucleic acid and the nucleic acid is comminuted by a nuclease.

56. The method as claimed in claim 54, wherein the substrate-molecule complex is moved to and fro during comminution.

57. The method as claimed in claim 42, wherein the released molecules are denatured.

58. The method as claimed in claim 57, wherein the molecules are denatured by an increase in temperature.

59. The method as claimed in claim 57, wherein the molecules are denatured chemically.

60. The method as claimed in claim 42, wherein only a prokaryotic nucleic acid is bound to the substrate-molecule complex by the substrate.

61. The method as claimed in claim 42, wherein the substrate-molecule complex is moved into the amplification chamber of the device and an amplification of the molecule is carried out.

62. The method as claimed in claim 61, wherein the amplification is performed by means of a PCR, wherein the PCR comprises at least one thermal cycle.

63. The method as claimed in claim 61, wherein the nucleic acid is an RNA and is reverse transcribed into cDNA prior to the amplification.

64. A method for analyzing a sample, comprising:
preparing the sample by a method as claimed in claims 42; and
detecting the molecule.

65. The method as claimed in claim 64, wherein the molecule is detected by detecting a binding of the molecule to the catcher probe.

66. The method as claimed in claim 64, wherein the substrate-molecule complex is moved to the catcher probe.

67. The method as claimed in claim 64, wherein the immobilized magnetic beads are detected for detecting the hybridization.

68. A method for preparing a sample, comprising:
binding the at least one biological structure of a sample;
using at least one magnetic field generator to move the at least one bound biological structure;
releasing the at least one biological molecule contained in the bound structure; and
binding the released at least one biological molecule.
